(19) United States
(12) Patent Application Publication
(10) Pub. No.: US 2018/0050047 A1
(43) Pub. Date: Feb. 22, 2018

(54) CORTEXOLONE 17alpha-PROPIONATE FOR USE IN THE TREATMENT OF SKIN WOUNDS AND/OR ATROPHIC SKIN DISORDERS

(71) Applicant: Cosmo Technologies Limited, Dublin 2 (IE)

(72) Inventors: Luigi MORO, Lainate (MI) (IT); Luigi Maria LONGO, Figino Serenza (CO) (IT); Giuseppe CELASCO, Lainate (MI) (IT)

(73) Assignee: Cosmo Technologies Limited, Dublin 2 (IE)

(21) Appl. No.: 15/793,011

(22) Filed: Oct. 25, 2017

Related U.S. Application Data
(63) Continuation of application No. 15/101,713, filed on Jun. 3, 2016.

(30) Foreign Application Priority Data
Dec. 20, 2013 (IT) MI2013A002157

(51) Int. Cl. A61K 31/573; A61K 9/14; A61K 9/10; A61K 9/12; A61K 9/08; A61K 9/06; A61K 9/00; A61K 47/40

(52) U.S. Cl. CPC A61K 31/573; A61K 9/14; A61K 9/10; A61K 9/122; A61K 9/12; A61K 47/40; A61K 9/08; A61K 9/06; A61K 9/0014

(57) ABSTRACT

The present invention provides cortexolone 17α-propionate for use in the treatment of skin wounds and/or atrophic skin disorders. The present invention also provides pharmaceutical or cosmetic compositions comprising cortexolone 17α-propionate for use in the treatment of skin wounds and/or of atrophic skin disorders.

### BACKGROUND OF THE INVENTION

Wounds are injuries to the body (as from violence, accident, or surgery) that typically involve laceration or breaking of a membrane (as the skin) and usually damage to underlying tissues. Wounds are also known to be injuries to the body caused by physical means with disruption of the normal continuity of body structures (Dorland's Illustrated Medical Dictionary, page 1549, 23th Edition, 1960).

In the wound healing process, the overlapping segments of the repair process are conceptually defined as inflammation, proliferation and remodeling. During the inflammatory phase, hemostasis occurs and an acute inflammatory infiltrate ensues. The proliferative phase is characterized by fibroplasias, granulation, contraction and epithelialization. The final phase is remodeling, which is commonly described as scar maturation. Chronic or non-healing wounds, such as ulcers, are loss of substance on a cutaneous or mucous surface, causing gradual disintegration and necrosis of the tissues (Dorland's Illustrated Medical Dictionary, page 1489, 23^{th} Edition, 1960).

Chronic or non-healing wounds are particular types of skin lesions, which are chronic open wounds that fail to proceed through an orderly and timely series of events to produce a durable, structural and functional closure, through a re-epithelization and a healing in a reasonable amount of time. Chronic wounds are clinically stagnant and may be present for months or even years. A particular type of chronic (or non-healing) wounds are the “trophic ulcers of the skin”, which are disabling skin disorders which include a group of lesions caused by faulty nutrition in the affected part of the skin. Trophic ulcers of the skin include, for example, decubitus ulcers (also known as pressure sores or bed sores), lower-extremity ulcers (also known as leg ulcers), diabetic ulcers, neuropathic ulcers, venous stasis ulcers, arterial ulcers, diabetic foot ulcers and the like. Taking into account the different mechanisms involved, the trophic ulcers of the skin can be grouped into the main following categories (Martindale, 36th ed. 2009, page 1585): decubitus ulcers (bedsores, or pressure sores), which occur in patients with extended immobility when a prolonged pressure on the skin over a bony prominence produces localized ischemia; leg ulcers (vascular ulcers), which can result from venous incompetence (varicose ulcers, post-phlebitic ulcers) or can be ischemic in origin (arterial ulcers); diabetic ulcers, which include also neuropathic ulcers and are closely related to dysfunction of the microcirculation of foot and to concomitant peripheral neuropathy. The prolonged impairment of local circulation is considered, in all above categories, the main pathogenetic factor leading to faulty nutrition of the skin. As a result, there is tissue hypoxia which impairs the viability of the derma, leading to dermal sclerosis, necrosis of fibroblasts and, at the end, skin ulceration. Burns are skin wounds caused for instance by the contact of heat, caustics, friction or electricity, which are classified according to the degree of the damage, as simple hyperemic, vesicant, destructive of skin and underlying tissues (Blackiston's New Gould Medical Dictionary, page 185, 2^{nd} Edition, 1956).

Similar to trophic ulcers of the skin, but with lowest degree of severity and involving mainly aesthetic aspects, are the so called atrophic skin disorders, induced by physical (i.e.: light, UV and ionizing radiations) or chemical (i.e.: free radicals, superoxide anion) factors. In these disorders the impairment of local circulation is not primarily involved, and there is no loss of tissue leading to ulceration of the skin, as in the case of the trophic ulcers. These disorders are produced by direct noxious effects on dermal cells, and are characterized by evident atrophy and thinning of the derma in which the fibroblasts are numerically reduced and the production of collagen, elastic and reticular fibres is impaired. These structural alterations lead to skin damages represented by drying, thinning, scaling and loose of elasticity.

There is a growing body of evidence that sex hormones influence wound repair processes. Elderly males heal wounds more slowly than elderly females and have reduced matrix deposition and an increased inflammatory response. In a study on a group of elderly males, increasing testosterone levels were linked to delayed wound healing. Androgen receptor (AR) expression is localized to keratinocites, inflammatory cells and fibroblasts during wound healing, suggesting that androgens may be involved in the regulation of inflammation and/or repair. Recent studies have suggested that, intriguingly, endogenous testosterone inhibits wound healing and promotes inflammation. In animal models, castrated male mice exhibit accelerated cutaneous wound healing compared to sham-operated controls accompanied by an attenuated inflammatory response, reduced macrophage invasion and increased collagen deposition.

WO03/014141 A1, herein incorporated by reference in its entirety, discloses 17α-monoesters, 21-monoesters and 17α,21-diesters of 17α,21-dihydroxy-pregna-4-ene-3,20-dione (also known in the art as cortexolone) and of 17α,21-dihydroxy-pregna-4,9(11)-diene-3,20-dione (also known in the art as 9,11-dehydrocortexolone) as antiandrogenic drugs, and the processes to obtain them.

WO2009/019138A2, herein incorporated by reference in its entirety, discloses an enzymatic process for the obtainment of 17-alpha monoesters of cortexolone and of 9,11-dehydrocortexolone; furthermore, WO2009/019138A2 discloses crystalline forms of cortexolone 17α-propionate, namely crystalline form I, form II, form III and hydrate form IV, and the processes to obtain them.

Chronic wounds, including venous, diabetic, and pressure ulcers, not only affect the quality of life but also represent a burden and enormous drain on financial and human resources. In developed countries, it has been estimated that 1% to 2% of the population will experience a chronic wound during their lifetime. In United States alone, chronic wounds affect 6.5 million patients. The burden of treating chronic wounds is growing rapidly due to increasing health care costs, an aging population and a sharp rise in the incidence of diabetes and obesity worldwide, particularly in the industrialized countries. In severe cases, mainly in cases of diabetic foot, amputation may become necessary to prevent the spreading of the necrosis.

There is therefore a growing need of improved therapies in the treatment of skin wounds and/or of atrophic skin disorders. In particular, there is a growing need of therapies capable of promoting the healing of wounds, particularly of chronic wounds, and/or the repair of atrophic skin which allow a self-medication at home and thus prevent or reduce the necessity of an hospitalization, which has an impact on the wellness of the patient and on the health care costs.

All publications, patents and patent applications cited herein, whether supra or infra, are hereby incorporated by reference in their entirety.

### BRIEF DESCRIPTION OF THE FIGURES

FIG. 1: a representation of the area of the skin wound selected for the measurements in the animal model.

FIG. 2: analysis of the intensity of proliferative activity of cortexolone 17α-propionate compound on skin wound healing in REPTOP mitolRE mice. Data are elaborated to measure photon-emission in wound areas as indicated in FIG. 1. Each bar represents the mean ratio versus time=0±SEM of photon-emission measured in 4 animals/group; (**p<0.01 vs time 0). No statistical significance was found between comparator (CICATRENE) and cortexolone 17α-propionate groups at any time point.

FIG. 3: skin wound area measurement. Each bar represents the mean±SEM of wound area measured in 4 animals/group; (***p<0.001 vs time 0; **p<0.01 vs time 0). No statistical significance was found between comparator (CICATRENE) and cortexolone 17α-propionate groups at any time point.

FIG. 4: in vivo skin wound healing bioluminescence imaging of REPTOP mitolRE mouse after indicated treatments. Photos portray whole body (dorsal view) at time 0, 1, 2, 3 and 6 days after treatments in an image processed to indicate the intensity of photon-emission in wound areas.

### SUMMARY OF THE INVENTION

The invention herein disclosed provides cortexolone 17α-propionate for use in the treatment of at least one skin wound and/or of at least one atrophic skin disorder. The cortexolone 17α-propionate for use in the treatment of at least one skin wound and/or of at least one atrophic skin disorder according to the invention is administered to a mammal. Said mammal is a human or an animal, preferably a human.

The invention herein disclosed also provides a method of treating at least one skin wound and/or at least one atrophic skin disorder, said method comprising the administration of cortexolone 17α-propionate to a mammal afflicted with at least one skin wound and/or with at least one atrophic skin disorder. Said mammal is a human or an animal, preferably a human.

The invention herein disclosed also provides a pharmaceutical or cosmetic composition comprising cortexolone 17α-propionate and at least one physiologically acceptable excipient for use in the treatment of at least one skin wound and/or of at least one atrophic skin disorder.

The pharmaceutical or cosmetic composition comprising cortexolone 17α-propionate and at least one physiologically acceptable excipient for use in the treatment of at least one skin wound and/or of at least one atrophic skin disorder according to the invention is administered to a mammal. Said mammal is a human or an animal, preferably a human.

The invention herein disclosed provides a method for treating at least one skin wound and/or at least atrophic skin disorder, wherein said method comprises the administration of a pharmaceutical or cosmetic composition comprising cortexolone 17α-propionate and at least one physiologically acceptable excipient to a mammal afflicted with at least one skin wound and/or with at least atrophic skin disorder. Said mammal is a human or an animal, preferably a human.

The treatment of the at least one skin wound according to the invention is preferably the treatment of a burn skin wound or the treatment of a throphic ulcer of the skin.

### DETAILED DESCRIPTION OF THE INVENTION

The scientific literature emphasizes the prolonged impairment of local circulation as the main pathogenetic factor leading to faulty nutrition of the skin, with a consequent tissue hypoxia which impairs the viability of the derma, leading to dermal sclerosis, necrosis of fibroblasts and, at the end, skin ulceration. In the recent years, several scientific data point out the role of sex hormones in wound repair processes, in particular the role of an excess of endogenous androgens (for example, testosterone and/or its metabolite, dihydrotestosterone) in suppressing or slowing down the wound healing process of the skin.

Some 17α-monoesters and/or 17α,21-diesters of cortexolone, a class of compounds provided with a strong local antiandrogenic activity, were tested to verify their possible therapeutic use in the treatment of skin wounds and/or of atrophic skin disorders. Said 17α-monoesters and/or 17α,21-diesters of cortexolone were tested in vitro to verify their efficacy on the dermal human fibroblasts in:

a) inducing an evident stimulating effect on human dermal fibroblast proliferation and migration;

b) promoting the extra-cellular protein synthesis; and/or

c) increasing the synthesis of type I procollagen, which is the precursor of the mature collagen.

As a person skilled in the art will recognize, the above fibroblast proliferation and migration, the extra-cellular protein synthesis and the synthesis of type I procollagen are main key processes in skin wound healing and/or atrophic skin repair. A person skilled in the art will further recognize that a compound having a pharmacological efficacy in promoting at least one of the above processes will be effective in accelerating the healing of a skin wound and/or the repair of an atrophic skin.

Three 17α-monoesters and/or 17α,21-diesters of cortexolone of formula (I), namely:

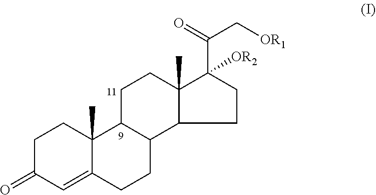

cortexolone 17α-propionate according to formula I, wherein R₁ is hydrogen and R₂ is propionyl;

cortexolone 17α-valerate according to formula I, wherein R1 is hydrogen and R2 is valeryl;

cortexolone 17α,21-dibutirrate according to formula I, wherein R1 and R2 are butiryl;

all of which provided with a local antiandrogenic activity, were tested to evaluate their efficacy in the hereinabove processes at points a) to c).

The local antiandrogenic activity of the above three compounds is reported in Table 1 below.

TABLE 1
Local antiandrogenic activity (measured by flank organ test in
hamster) of cortexolone 17α-propionate, cortexolone 17α-
valerate and cortexolone 17α,21-dibutyrate - Percent inhibition
of testosterone propionate (TP) response.

| Daily dose (test | | | |
|---|---|---|---|
| compound + TP) | Cortexolone | Cortexolone | Cortexolone |
| (μg) | 17α-propionate | 17α-valerate | 17α,21-dibutyrate |
| 100 + 4 | 62 | 66 | 84 |
| 200 + 4 | 69 | 65 | 89 |
| 400 + 4 | 86 | 73 | 97 |
| Mean inhibition (%) | 72 | 68 | 90 |

Table 1 reports the results of the antiandrogenic activity of cortexolone 17α-propionate, cortexolone 17α-valerate and cortexolone 17α,21-dibutyrate topically applied on the flank organ in hamster. The numbers indicate the percent inhibition of the stimulating response induced by the application of testosterone propionate (TP): the higher the number, the higher the inhibition of the androgenic response to testosterone propionate (TP).

The local antiandrogenic activity of cortexolone 17α-propionate and of cortexolone 17α-valerate is comparable, whilst cortexolone 17α,21-dibutyrate is more potent and has the highest inhibitory effect on the response induced by the application of testosterone propionate (TP), and also at the low dose the inhibition is more effective than with the other two compounds.

The effects of the same 17α-monoesters and/or 17α,21-diesters of cortexolone were tested on human dermal fibroblasts according to the following Examples 1-4. The results are summarized in Table 2 below.

TABLE 2
Effects of 17α-monoesters and/or 17α,21-diesters of
cortexolone on human dermal fibroblasts (Δ% vs. control).

| | | | | Cortexolone |
|---|---|---|---|---|
| | Conc. | Cortexolone | Cortexolone | 17α,21- |
| Activity | (μM) | 17α-propionate | 17α-valerate | dibutyrate |
| Fibroblast | 50 | 31.0 | −33.8 | 9.6 |
| proliferation (see | | | | |
| Example 1) | | | | |
| Protein synthesis | 50 | 27.4 | −3.8 | 0.6 |
| (see Example 3) | | | | |
| Procollagen I | 50 | 199.6 | −14.6 | −2.8 |
| Synthesis | | | | |
| (see Example 4) | | | | |

It was surprisingly found that only cortexolone 17α-propionate showed a strong and significant efficacy in:

a) inducing an evident stimulating effect on human dermal fibroblast proliferation;

b) promoting the extra-cellular protein synthesis; and

c) increasing the synthesis of type I procollagen, which is the precursor of the mature collagen.

The values reported in Table 2 demonstrate a significant effect of cortexolone 17α-propionate in stimulating the fibroblast proliferation and increasing the production of components of the extra-cellular matrix (evaluated in accordance to above processes a) to c)), which results in a consequent significant effect in the skin wound healing and/or in the repair of an atrophic skin. On the contrary, cortexolone 17α-valerate and/or cortexolone 17α,21-dibutyrate did not show any effect.

The results reported in the above Table 2 show that cortexolone 17α-valerate and cortexolone 17α,21-dibutyrate, despite their antiandrogenic activity equal to or superior to cortexolone 17α-propionate, have no effect with respect to the control in all the evaluated processes a) to c), while cortexolone 17α-propionate demonstrates a significantly higher effect with respect to the control and to the other tested antiandrogenic drugs in all the said processes a) to c).

The inhibition of the response to endogenous androgens (such as testosterone and/or dihydrotestosterone) cannot be therefore considered the mechanism responsible for the above mentioned effects on the dermal human fibroblasts. It was now surprisingly found that the properties of cortexolone 17α-propionate on the dermal human fibroblasts are not in relationship with the local antiandrogenic activity of the compound, considering that compounds belonging to the same chemical family of formula (I) and provided with similar or higher antiandrogenic activity (i.e. cortexolone 17α-valerate and cortexolone 17α,21-dibutyrate) are devoid of such effects.

One aspect of the invention herein disclosed provides cortexolone 17α-propionate for use in the treatment of at least one skin wound and/or of at least one atrophic skin disorder. Cortexolone 17α-propionate according to the invention is preferably administered to a mammal.

Another aspect of the invention herein disclosed provides a method of treating at least one skin wound and/or of at least one atrophic skin disorder, said method comprising the administration of cortexolone 17α-propionate to a mammal afflicted with at least one skin wound and/or with at least one atrophic skin disorder.

According to the invention said mammal is a human or an animal, preferably a human. According to the invention herein disclosed, when said mammal is an animal it can be preferably selected from a canid, a feline, a bovine, a bovid, an ovine, an equine and/or a swine (such as, dogs, cats, cows, goats, sheeps, horses, pigs and/or the like).

According to the invention, the treatment of the at least one skin wound is preferably the treatment of a burn skin wound or the treatment of a throphic ulcer of the skin.

According to the invention, healing of at least one skin wound refers to inducing and/or promoting repair of a wound comprising, in a non-limiting way, arresting tissue damage such as necrotization, promoting epidermal and/or dermal tissue growth and repair. According to the invention, repair of at least one atrophic skin disorder refers to inducing and/or promoting repair of an atrophic skin disorder comprising, in a non-limiting way, arresting tissue damage such as dermal atrophy, thinning and necrosis, and promoting epidermal and/or dermal tissue growth and repair.

According to the invention herein disclosed, said at least one skin wound can be selected in the group comprising, but not limited to: open skin wounds, burn skin wounds, chronic skin wounds (also known as non-healing skin wounds) and/or the like (Fletcher J. Nursing Standard, February 20, Vol. 22, no 24, pages 62-68, 2008 and NSCCAHS Wound Assessment Guidelines, Northern Sydney Central Coast, 18 Nov. 2008)).

According to the invention herein disclosed and detailed in the following definitions section, exemplary open skin wounds comprise, in a non-limiting way: incisions, incised wounds, surgical wounds, lacerations, abrasions, puncture wounds, bite wounds, scratch wounds, penetration wounds, gunshot wounds, avulsions, blisters, and/or the like.

According to the invention herein disclosed, and detailed in the following definitions section, exemplary burn skin wounds comprise, in a non-limiting way: burn wounds caused by heat, burn wounds caused by friction, burn wounds caused by electricity, bum wounds caused by chemicals, burn wounds caused by radiation and/or the like.

According to the invention herein disclosed, and detailed in the following definitions section, exemplary chronic skin wounds (also known as non-healing skin wounds) comprise, in a non-limiting way: cutaneous ulcers, trophic ulcers of the skin, radiation injuries, chronic ulcers in elderly humans (aging defects) and/or the like. Said trophic ulcers of the skin are wounds or lesions caused by faulty nutrition in the affected part and may be selected in the group comprising, in a non-limiting way: decubitus ulcers (also known as pressure sores or bed sores), lower-extremity ulcers (also known as leg ulcers), diabetic ulcers, neuropathic ulcers, venous stasis ulcers, arterial ulcers, diabetic foot ulcers and/or the like. In a preferred embodiment of the invention, said skin wound is an open skin wound. In another preferred embodiment of the invention, said skin wound is a burn skin wound. In another preferred embodiment, said skin wound is a chronic skin wound (also known as non-healing skin wound) and in more preferred embodiment it is a trophic ulcer of the skin.

According to the invention herein disclosed, said at least one atrophic skin disorder can be selected in the group comprising, in a non-limiting way: skin ageing, photo-ageing, wrinkles, lines, dermatomyositis, atrophic striae, radiation dermatitis, scars, acrodermatitis, anetoderma and the like.

In a preferred embodiment, said atrophic skin disorder is anetoderma. In another preferred embodiment of the invention, said atrophic skin disorder is skin aging. In another preferred embodiment, said atrophic skin disorder is photo-aging. In another preferred embodiment, said atrophic skin disorder is a wrinkle or a line. According to the invention, any known crystalline or non-crystalline form of cortexolone 17α-propionate can be used.

According to an embodiment of the invention, crystalline form of cortexolone 17α-propionate can be represented by crystalline form I, crystalline form II, crystalline form III and/or crystalline hydrate form IV of cortexolone 17α-propionate as disclosed in WO2009/019138 A2, herein incorporated by reference in its entirety for all that it discloses.

According to the invention, cortexolone 17α-propionate is preferably administered to a mammal, said mammal being a human or an animal; more preferably, said mammal is a human.

According to the invention herein disclosed, when said mammal is an animal it can be preferably selected from a canid, a feline, a bovine, a bovid, an ovine, an equine and/or a swine (such as, dogs, cats, cows, goats, sheeps, horses, pigs and/or the like).

According to the invention, cortexolone 17α-propionate is preferably administered topically.

According to the invention, cortexolone 17α-propionate is preferably administered in form of a pharmaceutical or cosmetic composition, as defined below.

Cortexolone 17α-propionate can be combined with at least one physiologically acceptable excipient to obtain a pharmaceutical or cosmetic composition, preferably to be topically applied on the wounded skin surface and/or on the atrophic skin and/or on a portion thereof.

More preferably, cortexolone 17α-propionate of the invention can be formulated in solid, semi-solid, pasty or liquid form. Exemplary solid, semi-solid, pasty or liquid forms comprise, in a non-limiting way: powders, freeze-dried powders, solutions, emulsions, gels (such as hydrogels, anhydrous gels and/or lipogels), pastes, creams, ointments, lotions, suspensions, sprays, pressurized-sprays, plasters, gauzes, medicated gauzes and the like. Preferably, cortexolone 17α-propionate of the invention is formulated in form of gels (such as hydrogels, anhydrous gels and/or lipogels), ointments, creams, solutions, sprays, pressurized-sprays, plasters, gauzes, medicates gauzes, freeze-dried powders or powders. More preferably, cortexolone 17α-propionate of the invention is formulated in form of sprays, pressurized sprays, ointments, gels (such as hydrogels, anhydrous gels and/or lipogels), powders or medicated gauzes.

Accordingly, a further aspect of the invention herein disclosed provides a pharmaceutical or cosmetic composition comprising cortexolone 17α-propionate and at least one physiologically acceptable excipient for use in the treatment of at least one skin wound and/or of at least one atrophic skin disorder.

According to the invention, said pharmaceutical or cosmetic composition comprising cortexolone 17α-propionate and at least one physiologically acceptable excipient is administered to a mammal. Said mammal is a human or an animal, preferably a human.

A further aspect of the invention herein disclosed provides a method of treating at least one wound or at least one atrophic skin disorder, wherein said method comprises the administration of a pharmaceutical or cosmetic composition comprising cortexolone 17α-propionate and at least one physiologically acceptable excipient to a mammal afflicted with at least one skin wound and/or with at least one atrophic skin disorder. According to the invention, said mammal is a human or an animal, preferably a human. According to the invention herein disclosed, when said mammal is an animal it can be preferably selected from a canid, a feline, a bovine, a bovid, an ovine, an equine and/or a swine (such as, dogs, cats, cows, goats, sheeps, horses, pigs and/or the like).

According to the invention, the treatment of at the least one skin wound is preferably the treatment of a burn skin wound or the treatment of a throphic ulcer of the skin.

According to the invention herein disclosed, any known crystalline or noncrystalline form of cortexolone 17α-propionate can be used as starting material for the preparation of said pharmaceutical or cosmetic composition.

According to an embodiment of the invention, the crystalline form of cortexolone 17α-propionate can be represented by crystalline form I, crystalline form II, crystalline form III and/or crystalline hydrate form IV of cortexolone 17α-propionate as disclosed in WO2009/019138 A2, herein incorporated by reference in its entirety for all that it discloses.

According to an embodiment, said pharmaceutical or cosmetic composition comprises the whole amount of cortexolone 17α-propionate in dissolved form into the vehicle. According to such an embodiment, at the end of the manufacturing process of said pharmaceutical or cosmetic composition a crystalline form of cortexolone 17α-propionate is not recognizable, since said cortexolone 17α-propionate is wholly dissolved and thus no crystals are present.

According to another embodiment, said pharmaceutical or cosmetic composition comprises the whole amount of cortexolone 17α-propionate in dispersed solid state. In further another embodiment, said pharmaceutical composition comprises a part of the total amount of cortexolone 17α-propionate in dispersed solid state, and a part of the total amount of cortexolone 17α-propionate in dissolved form into the vehicle.

According to an embodiment, the crystalline form of cortexolone 17α-propionate starting material is maintained throughout the manufacturing process of said pharmaceutical or cosmetic composition, thus the crystalline form of said cortexolone 17α-propionate in dispersed solid state in said pharmaceutical or cosmetic composition is the same of cortexolone 17α-propionate starting material. In another embodiment, the crystalline form of cortexolone 17α-propionate starting material changes during the manufacturing process of said pharmaceutical or cosmetic composition as a consequence of said manufacturing process, thus the crystalline form of said cortexolone 17α-propionate in dispersed solid state in said pharmaceutical or cosmetic composition is different from that of cortexolone 17α-propionate starting material.

Pharmaceutical or cosmetic compositions of the invention herein disclosed are preferably topical compositions.

Pharmaceutical or cosmetic compositions of the invention herein disclosed are preferably formulated in solid, semi-solid, pasty or liquid form. Exemplary pharmaceutical or cosmetic compositions comprise, in a non-limiting way: powders, freeze-dried powders, solutions, emulsions, gels (such as hydrogels, anhydrous gels and/or lipogels), pastes, creams, ointments, lotions, suspensions, sprays, pressurized sprays, plasters, gauzes, medicated gauzes and the like. Preferably, such pharmaceutical or cosmetic compositions are gels (such as hydrogels, anhydrous gels and/or lipogels), ointments, creams, solutions, sprays, pressurized sprays, plasters, gauzes, medicated gauzes, powders or freeze-dried powders. More preferably, such pharmaceutical or cosmetic compositions are sprays, pressurized sprays, ointments, gels (such as hydrogels, anhydrous gels and/or lipogels), powders or medicated gauzes.

In an embodiment, said pharmaceutical or cosmetic composition for use in the treatment of at least one skin wound and/or at least one atrophic skin disorder comprises:

a) cortexolone 17α-propionate;

b) at least one physiologically acceptable excipient.

According to the invention herein disclosed, said physiologically acceptable excipient can be any non-toxic, biocompatible, auxiliary substance conventionally usable to formulate a mixture aimed to allow the topical administration of pharmaceuticals or cosmetics when applied directly to the skin, in which cortexolone 17α-propionate remains stable and bioavailable.

Suitable physiologically acceptable pharmaceutical or cosmetic excipients are well known in the art, and may be selected from the groups comprising, in a non-limiting way: adsorbents for powder compositions, diluents, freeze-drying agents, fillers, glidants, lubricants, solvents, cosolvents, oleaginous vehicles, emollients, ointment bases, emulsifying agents and surfactants, emulsion stabilizing agents, skin softening agents, gel bases and gelling agents, thickening agents, viscosity-increasing agents, rheology modifiers, dispersing agents, dissolution enhancers, stabilizing agents, stiffening agents, buffering agents, wetting agents, chelating agents, antimicrobial preservatives, acidifying agents, alkalizing agents and the like. Any mixture of said physiologically acceptable excipients can be used according to the invention.

The amount of cortexolone 17α-propionate in said pharmaceutical or cosmetic composition, preferably topical pharmaceutical or cosmetic composition, is such that an effective dosage level can be obtained upon administration to a mammal suffering of at least one skin wound and/or of at least one atrophic skin disorder. According to an embodiment, said pharmaceutical or cosmetic composition is in solid form and comprises cortexolone 17α-propionate in an amount ranging from about 0.1% w/w to about 80% w/w, preferably from about 1% w/w to about 75% w/w, more preferably from about 5% w/w to about 70% w/w, much more preferably from about 10% w/w to about 60% w/w, with respect to the total weight of the composition.

According to an embodiment, said pharmaceutical or cosmetic composition in solid form comprises cortexolone 17α-propionate in an amount of about 60% w/w, with respect to the total weight of the composition.

According to an embodiment, said pharmaceutical or cosmetic composition in solid form comprises cortexolone 17α-propionate in an amount of about 50% w/w, with respect to the total weight of the composition.

According to an embodiment, said pharmaceutical or cosmetic composition in solid form comprises cortexolone 17α-propionate in an amount of about 40% w/w, with respect to the total weight of the composition.

According to an embodiment, said pharmaceutical or cosmetic composition in solid form comprises cortexolone 17α-propionate in an amount of about 30% w/w, with respect to the total weight of the composition.

According to an embodiment, said pharmaceutical or cosmetic composition in solid form comprises cortexolone 17α-propionate in an amount of about 25% w/w, with respect to the total weight of the composition.

According to an embodiment, said pharmaceutical or cosmetic composition in solid form comprises cortexolone 17α-propionate in an amount of about 20% w/w, with respect to the total weight of the composition.

According to an embodiment, said pharmaceutical or cosmetic composition in solid form comprises cortexolone 17α-propionate in an amount of about 15% w/w, with respect to the total weight of the composition.

According to an embodiment, said pharmaceutical or cosmetic composition in solid form comprises cortexolone 17α-propionate in an amount of about 10% w/w, with respect to the total weight of the composition.

According to an embodiment, said pharmaceutical or cosmetic composition in solid form comprises cortexolone 17α-propionate in an amount of about 5% w/w, with respect to the total weight of the composition.

According to an embodiment, said pharmaceutical or cosmetic composition in solid form comprises cortexolone 17α-propionate in an amount of about 3% w/w, with respect to the total weight of the composition. According to an embodiment, said pharmaceutical or cosmetic composition in solid form comprises cortexolone 17α-propionate in an amount of about 1% w/w, with respect to the total weight of the composition.

According to some embodiments, said pharmaceutical or cosmetic composition is in liquid, pasty or semi-solid form and comprises cortexolone 17α-propionate in an amount ranging from about 0.1% w/w to about 50% w/w, preferably from about 0.2% w/w to about 30% w/w, more preferably from about 0.5% w/w to about 20% w/w, much more preferably from about 1% w/w to about 15% w/w, with respect to the total weight of the composition.

According to some embodiments, said pharmaceutical or cosmetic composition in liquid, pasty or semi-solid form comprises cortexolone 17α-propionate in an amount of about 15% w/w, with respect to the total weight of the composition.

According to some embodiments, said pharmaceutical or cosmetic composition in liquid, pasty or semi-solid form comprises cortexolone 17α-propionate in an amount of about 15% w/w, with respect to the total weight of the composition.

According to some embodiments, said pharmaceutical or cosmetic composition in liquid, pasty or semi-solid form comprises cortexolone 17α-propionate in an amount of about 5% w/w, with respect to the total weight of the composition.

According to some embodiments, said pharmaceutical or cosmetic composition in liquid, pasty or semi-solid form comprises cortexolone 17α-propionate in an amount of about 3% w/w, with respect to the total weight of the composition.

According to some embodiments, said pharmaceutical or cosmetic composition in liquid, pasty or semi-solid form comprises cortexolone 17α-propionate in an amount of about 2% w/w, with respect to the total weight of the composition.

According to some embodiments, said pharmaceutical or cosmetic composition in liquid, pasty or semi-solid form comprises cortexolone 17α-propionate in an amount of about 1% w/w, with respect to the total weight of the composition.

All publications, patents and patent applications cited herein, whether supra or infra, are hereby incorporated by reference in their entirety.

### Definitions

References in the specification to “one embodiment”, “an embodiment” and similar indicate that the described embodiment may include a particular aspect, feature, structure or characteristic. Moreover, such phrases may, but do not necessarily, refer to the same embodiment referred to in other portions of the specification. Further, when a particular aspect, feature, structure or characteristic is described in connection with an embodiment, it is within knowledge of a person skilled in the art to affect or connect said aspect, feature, structure or characteristic with other embodiments, whether or not explicitly described.

The singular forms “a”, “an” and “the” include plural references unless the context clearly dictates otherwise. Thus, for example, a reference to “a compound” includes a plurality of such compounds; in the same way, a mammal afflicted with “a skin wound” means that said mammal has at least one skin wound. It is further noted that the claims may be drafted to exclude any optional element. As such, this statement is intended to serve as antecedent basis for the use of exclusive terminology, such as “solely”, “only”, and the like, in connection with the recitation of claims elements or use of a “negative” limitation.

The term “and/or” means anyone of the items, any combination of the items, or all the items with which this term is associated.

Unless indicated otherwise herein, the term “about” is intended to include values, e.g. weight percentages, proximate to the recited range that are equivalent in terms of the functionality of the individual ingredient, the composition, or the embodiment.

A person skilled in the art will recognize that, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range.

A person skilled in the art will recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of anyone or more of members of a recited group. Accordingly, provisos may apply to any of the disclosed categories or embodiments whereby anyone or more of the recited elements, species, or embodiments, may be excluded from such categories or embodiments, for example, as used in an explicit negative limitation.

The term “an effective amount” or “effective dosage level” refers to an amount effective to treat a disease, a disorder and/or a condition, of to bring about a recited effect. For example, an amount effective or an effective dosage can be an amount effective to reduce the progression or severity of the condition or symptoms to be treated. Determination of a therapeutically effective amount is well within the capacity of persons skilled in the art. The term “an effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effects.

The compositions and uses/methods described herein can be used for aiding skin wound healing.

The term “wound healing” refers to the process inducing and/or promoting repair of a wound comprising, in a non-limiting way, arresting tissue damage such as necrotization, promoting epidermal and/or dermal tissue growth and repair. The term can further include reducing or eliminating the sensation of pain and discomfort attributable to a wound. The compositions and methods described herein can be used for aiding the repair of atrophic skin disorders. The term “atropic skin repair” refers to the process inducing and/or promoting the repair of an atrophic skin disorder comprising, in a non-limiting way, arresting tissue damage such as dermal atrophy, thinning and necrosis, and promoting tissue growth and repair. The term “wound” refers to an injury to the body that typically involves laceration or breaking of a membrane (as the skin) and usually damage to underlying tissues. A “skin wound” is a type of injury in which skin is torn, cut, or punctured (an “open wound”), or where blunt force trauma causes a contusion (a “closed wound”). In pathology, it specifically refers to a sharp injury which damages the dermis of the skin, said injury due to trauma, violence, accident or surgery. Open skin wounds can be classified according to the object that caused the wound. Exemplary types of open skin wounds comprise: incisions or incised wounds, caused by a clean, sharp-edged object such as a knife, razor, or glass splinter, such as surgical wounds; lacerations, irregular tear-like wounds caused by some blunt trauma; abrasions (grazes), superficial wounds in which the topmost layer of the skin (the epidermis) is scraped off; puncture wounds, caused by an object puncturing the skin, such as a nail or needle; bite wounds; scratch wounds; penetration wounds, caused by an object such as a knife entering and coming out from the skin; gunshot wounds, caused by a bullet or similar projectile driving into or through the body; avulsions, surface traumas where all layers of the skin have been torn away, exposing the underlying structures (i.e.—subcutaneous tissue, muscle, tendons, or bone); blisters, small pockets of fluid within the upper layers of the skin, typically caused by forceful rubbing (friction), burning, freezing, chemical exposure or infection. Another type of skin wounds are the burn wounds, which are injury to tissues caused by the contact with heat, flame, chemicals, electricity, or radiation. Exemplary types of burn wounds comprise: burn wounds caused by heat, burn wounds caused by friction, burn wounds caused by electricity, burn wounds caused by chemicals and burn wounds caused by radiation.

The terms “chronic skin wounds” or “non-healing skin wounds” mean particular types of skin lesions, which are chronic open wounds that fail to proceed through an orderly and timely series of events to produce a durable, structural and functional closure, through a re-epithelization and a healing in a reasonable amount of time. Said chronic wounds are clinically stagnant and may be present for months or even years. These lesions are characterized by a break in skin with loss of surface tissue, disintegration and necrosis of epithelial and dermal tissues, often accompanied by inflammation and/or microbial infection. Exemplary types of chronic or non-healing wounds comprise: cutaneous ulcers, trophic ulcers of the skin, radiation injuries, chronic ulcers in elderly humans (aging defect) and the like. Trophic ulcers of the skin are ulcers caused by faulty nutrition in the affected part. Exemplary types of trophic ulcers of the skin comprise, in a non-limiting way: decubitus ulcers (also known as pressure sores or pressure sores or bed sores), lower-extremity ulcers (also known as leg ulcers), diabetic ulcers, neuropathic ulcers, venous stasis ulcers, arterial ulcers, diabetic foot ulcers, radiation injuries, chronic ulcers in elderly humans (aging defects) and the like. “Decubitus ulcers”, also known as pressure sores or bed sores, develop over a bony prominence, usually in immobile patients, with the development of localized ischemia; the sacrum, ischium, and greater trochanter are the most common locations affected.

“Lower-extremity ulcers”, also known as leg ulcers, generally arise from either one of two vascular diseases: arterial or venous insufficiency. Most result from venous valvular disease.

“Diabetic ulcers”, which include “diabetic foot ulcers”, occur as a result of various factors, such as mechanical changes in conformation of the bony architecture of the foot, peripheral neuropathy, and atherosclerotic peripheral arterial disease, all of which occur with higher frequency and intensity in the diabetic population. Diabetic foot ulcers are responsible for more hospitalizations than any other complication of diabetes. Diabetes is the leading cause of nontraumatic lower extremity amputations in the United States, with approximately 5% of diabetics developing foot ulcers each year and 1% requiring amputation.

“Neuropathic ulcers” are related to the loss of protective sensation in the feet and legs as a result of a primary neurological condition, metabolic disease process (e.g., diabetes and/or renal failure), trauma, or surgery.

“Venous stasis ulcers” are wounds that are thought to occur due to improper functioning of venous valves, and usually are located in the legs. Leaky valves, obstructions, or regurgitation disturbs the flow of blood from the lower extremities back to the heart. The blood collects in the lower leg, damaging the tissues and causing wounds.

“Arterial ulcers” are due to a reduced arterial blood supply to the lower limb. The most common cause is atherosclerotic disease of the medium and large sized arteries. Other causes include diabetes, thromboangiitis, vasculitis, pyoderma gangrenosum, thalassaemia, and sickle cell disease. Further damage to the arterial system occurs with concurrent hypertension through damage of the intimal layer of the artery. The reduction in arterial blood supply results in tissue hypoxia and tissue damage.

“Radiation injuries” are caused by ionizing radiation emitted by sources such as the sun, x-ray and other diagnostic and/or therapeutic machines, tanning beds, and radioactive elements. External beam radiation through the skin has both acute and chronic effects on the skin. Acutely, a self-limiting erythema may develop that spontaneously resolves. Its late effect can be a more significant injury to fibroblasts, keratinocytes, and endothelial cells. DNA damage to these cells propagates over time and impairs the ability of these cells to divide successfully. Ultimately, a skin ulcer may occur spontaneously, but usually it occurs after repeated mild trauma such as abrasions.

The term “atrophic skin disorders” refers to disorders that are produced by direct noxious effects on dermal cells, and are characterized by evident atrophy and thinning of the derma, in which the fibroblasts are numerically reduced and the production of collagen, elastic and reticular fibres is impaired. These structural alterations lead to skin damages represented by drying, thinning, scaling and loose of elasticity. The atrophic skin disorders may be caused either by physical (i.e.: light, UV and ionizing radiations) or chemical (i.e.: free radicals, superoxide anion) factors, as well as they may be a consequence of a pathology such as lupus erythematosus. Exemplary atrophic skin disorders comprise: skin ageing, photo-ageing, wrinkles, lines, dermatomyositis, atrophic striae, radiation dermatitis, scars, acrodermatitis, anetoderma and the like.

The terms “comprising”, “having”, “including” and “containing” are to be construed as open-ended terms (i.e. meaning “including, but not limited to”) and are to be considered as providing support also for terms as “consist essentially of, “consisting essentially of, “consist of or “consisting of.

The terms “consist essentially of, “consisting essentially of are to be construed as a semi-closed terms, meaning that no other ingredients which materially affects the basic and novel characteristics (and optionally physiologically acceptable excipients and/or adjuvants) of the invention are included.

The terms “consists of, “consisting of are to be construed as a closed term.

The following examples are meant for clarifying the invention, without entailing any restrictions whatsoever with respect thereto.

### Examples

### Example 1: In-Vitro Fibroblast Proliferation

The activity of cortexolone 17α-propionate as wound repairing agent has been proven in an experiment where the drug has been tested in comparison with a known fibroblast proliferation promoter, the recombinant human Epidermal Growth Factor (rhEGF), according to the experimental design here below detailed.

Fibroblast proliferation: Adult human dermal fibroblasts (HDF) were appropriately grown, and were employed between 2nd and 6th passage. The fibroblasts, plated in a 96-wells plate at density of 5×10⁴ cells/mL, were incubated for 72 hours with recombinant human Epidermal Growth Factor (rhEGF, 100 nM) as positive control, or with cortexolone 17α-propionate at concentration of 1 μM, 5 μM, 25 μM, 50 μM, or with Cortisol 50 μM. Twenty-four hours before end of incubation, the cells were labeled with Bromo deoxyuridine (BrdU, 10 μL/well) and its incorporation into DNA was evaluated measuring the absorbance of the samples in an ELISA reader at 450 nm. The proliferation rate of the fibroblasts was evaluated on the increase of the absorbance of the samples. The results are reported in the Table 3 below.

TABLE 3
In-vitro fibroblast proliferation

| | | | | Δ % | |
|---|---|---|---|---|---|
| | | Absorbance ± SD | | vs. | |
| Treatment | Dose | Control | Treated | control | P = * |
| rhEGF | 100 nM | 1.88 ± 0.16 | 2.72 ± 0.22 | 49.49 | <0.001 |
| Cortexolone | 1 μM | 2.08 ± 0.19 | 2.06 ± 0.15 | −0.95 | 0.897 |
| 17α- | 5 μM | 1.92 ± 0.23 | 2.05 ± 0.24 | 6.71 | 0.618 |
| propionate | 25 μM | 1.96 ± 0.19 | 2.09 ± 0.19 | 7.00 | 0.127 |
| | 50 μM | 1.89 ± 0.19 | 2.48 ± 0.23 | 30.98 | <0.001 |
| Cortisol | 50 μM | 1.80 ± 0.10 | 0.78 ± 0.21 | −56.84 | <0.001 |

*t-test vs. relevant control

rhEGF, as expected, induced a significant increase of the absorbance, meaning proliferative effect, as compared to the relevant control (49.49%, P<0.001). Cortexolone 17α-propionate showed a dose-proportional proliferative stimulating effect starting from the dose of 5 μM (6.71%) and approaching to the effect produced by rhEGF, when administered at the dose of 50 μM (30.98%, P<0.001). On the contrary, cortisol strongly inhibited the fibroblasts proliferation (−56.84%, P<0.001).

The same test, in the same experimental conditions, was performed using cortexolone 17α-valerate and cortexolone 17α,21-dibutyrate, which did not show any proliferative stimulating effect, as shown in Table 2.

### Example 2: In-Vitro Fibroblast Migration

The activity of cortexolone 17α-propionate as wound repairing agent has been confirmed in an experiment where the drug has been tested in comparison with a known fibroblast migration promoter, the recombinant human Epidermal Growth Factor (rhEGF), according to the experimental design here below detailed.

Fibroblast migration: Adult human dermal fibroblasts (HDF) were appropriately grown, and were employed between 2^{nd} and 6^{th} passage. The cells were plated in suitable Culture-insert μ-Dish 35 mm, and were incubated for 24 h with vehicle alone, or with rhEGF 100 nM, or with cortexolone 17α-propionate 10 μM and 50 μM, or with Cortisol 50 μM. After incubation the culture-insert was removed and the cells were maintained in culture for further 24 hours. Immediately after insert removal, and at interval of 6 and 24 h, the plates were photographed by Digital Microscope Eyepiece. The entity of cell migration was measured using image analyzing software Image J, and was expressed as image density. The migration rate of the fibroblasts is reported in the Table 4 below.

TABLE 4
In-vitro fibroblast migration

| | | | | | Increase | Δ % |
|---|---|---|---|---|---|---|
| | | Absorbance ± SD | | | from | vs. |
| Treatment | Dose | t0 | 6 h | 24 h | t0-24 h | vehicle |
| Vehicle | — | 20.1 ± 1.8 | 21.5 ± 0.7 | 47.1 ± 3-0 | 27.0 | — |
| rhEGF | 100 nM | 22.1 ± 2.9 | 21.6 ± 1.0 | 65.7 ± 4.2 | 43.6 | 61.5 |
| | | | | (P = 0.003) | | |
| Cortexolone | 10 μM | 19.3 ± 0.8 | 24.0 ± 2.4 | 54.5 ± 2.5 | 35.2 | 30.3 |
| 17α-propionate | | | | (P = 0.030) | | |
| | 50 μM | 20.1 ± 2.0 | 25.1 ± 1.2 | 64.1 ± 5.6 | 44.0 | 62.9 |
| | | | | (P = 0.010) | | |
| Cortisol | 50 μM | 22.2 ± 3.2 | 22.9 ± 2.7 | 56.6 ± 4.4 | 34.4 | 27.4 |
| | | | | (P = 0.036) | | |

Cortexolone 17α-propionate enhanced significantly the fibroblast migration (30.3% to 62.9%) proportionally to the concentrations. This effect was very close to the effect elicited by rhEGF (61.5%). The effect of Cortisol (27.4%) was lower than that of that obtained with the lowest concentration of cortexolone 17α-propionate.

The results of the tests detailed in Example 1 and 2 demonstrate that cortexolone 17α-propionate is endowed with evident and robust effects on human dermal fibroblast proliferation and migration, confirming its beneficial role in wound healing processes and/or atrophic skin healing processes.

### Example 3: In-Vitro Effect on Extra-Cellular Protein Synthesis

The activity of cortexolone 17α-propionate as wound repairing agent has been confirmed in an experiment where the drug has been tested in comparison with a known extra-cellular protein synthesis promoter, the recombinant human Epidermal Growth Factor (rhEGF), according to the experimental design here below detailed

Adult human dermal fibroblasts (HDF) were appropriately grown, and were employed between 2nd and 6th passage. The fibroblasts, plated in a 24-wells plate at the density of 12×10⁴ cells/mL (1 mL/well), were incubated for 72 hours with rhEGF (100 nM) as positive control, or with cortexolone 17α-propionate at concentration of 5 μM, 25 μM, 50 μM. After 72h of incubation the supernatant were mixed with 1 mL of Bradford reagent and incubated for 45 minutes at room temperature. Each sample was then read in a spectrophotometer at 595 nm. The results of protein concentration are reported in the Table 5 below:

TABLE 5
Effect on extracellular protein synthesis

| | | | | Δ % | |
|---|---|---|---|---|---|
| | | Protein concentration | | vs. | |
| | | (mg/mL) | | con- | |
| Treatment | Dose | Control | Treated | trol | P = * |
| rhEGF | 100 nM | 0.654 ± 0.063 | 0.978 ± 0.074 | 49.54 | 0.002 |
| Cortexolone | 5 μM | 0.5 ± 0.010 | 0.5 ± 0.056 | 0 | 0.94 |
| 17α- | 25 μM | 0.788 ± 0.041 | 0.884 ± 0.028 | 12.18 | 0.15 |
| propionate | 50 μM | 0.664 ± 0.076 | 0.846 ± 0.087 | 27.41 | 0.15 |

* t-test vs. relevant control

In agreement with the stimulation of fibroblast proliferation (see Example 2, Table 3), the protein concentration increased after incubation with rhEGF (49.54%, P=0.002), and also with cortexolone 17α-propionate 25 μM (12,18%) and 50 μM (27.41%). These results clearly demonstrate that cortexolone 17α-propionate is also able to induce increase of extracellular protein synthesis, therefore actively contributing to skin wound healing and/or atrophic skin healing. The same test in the same experimental conditions was performed using cortexolone 17α-valerate and cortexolone 17α,21-dibutyrate, which did not show any effect in increasing the extracellular protein synthesis, as shown in Table 2.

### Example 4: In-Vitro Effect on Synthesis of Type I Procollagen

The activity of cortexolone 17α-propionate as wound repairing agent has been confirmed in an experiment where the drug has been tested in comparison with a known promoter of the synthesis of type I procollagen, like L-Ascorbic acid (Vitamin C), according to the experimental design here below detailed.

Adult human dermal fibroblasts (HDF) were appropriately grown, and were employed between 2nd and 4th passage. The fibroblasts, plated in a 24-wells plate at the density of 12×10⁴ cells/mL (1 mL/well), were incubated for 48 hours with L-Ascorbic acid (Vitamin C, 0.2 mM) as positive control, or with cortexolone 17α-propionate at concentration of 10 μM and 50 μM, or with Cortisol 50 μM. After the incubation the supernatant were collected and dosed for the terminal peptide of type 1 procollagen (PIP) with ELISA kit. The measurement of PIP concentration in the culture medium is representative for the production of collagen type I. The results of type I procollagen concentration (PIP) are reported in the Table 6 below:

TABLE 6
In-vitro effect on type I procollagen synthesis

| | | | | Δ % vs. | |
|---|---|---|---|---|---|
| Treatment | Dose | | | control | P = * |
| Control | — | | 1035.08 ± 215.47 | — | — |
| Vit. C | 0.2 | mM | 3735.27 ± 515.52 | 260.87 | <0.001 |
| Cortexolone | 10 | μM | 949.50 ± 149.81 | −8.27 | 0.629 |
| 17α-propionate | 50 | μM | 3100.98 ± 152.59 | 199.59 | <0.001 |
| Cortisol | 50 | μM | 259.36 | −74.94 | na |

* t-test

Cortexolone 17α-propionate induced an evident and robust increase of type I procollagen synthesis in comparison to untreated fibroblasts (199.59%, P<0.001). This effect resulted similar to the effect produced by Vitamin C (260.87%, P<0.001). Notably, Cortisol at concentration of 50 μM strongly reduced the procollagen I synthesis (−74.94%). The same test in the same experimental conditions was performed using cortexolone 17α-valerate and cortexolone 17α,21-dibutyrate, which did not show any effect in increasing the synthesis of type I procollagen, as shown in Table 2.

### Example 5: In Vivo Test of Wound Healing Activity of Cortexolone 17α-Propionate

The wound healing activity of the cortexolone 17α-propionate on cutaneous wounds was evaluated in an in vivo test foreseeing the use of REPTOP mitolRE mouse model. REPTOP mitolRE mice from TOP s.r.l. are engineered for the ubiquitous and proliferation-regulated expression of luciferase in proliferating tissues by embedding the transgene containing the minimal promoter of Cyclin B2d driving the firefly luciferase gene within HS4 insulator sequences. In this mouse, it has been demonstrated that photon emission is directly proportional to cell proliferation.

Cortexolone 17α-propionate was dissolved in 0.4% (v/v) tween 80 and 0.5% (w/v) carboxymethylcellulose in normal saline.

The test was carried out comparing the wound healing activity of cortexolone 17α-propionate at two different dosages (50 mg/mouse and 250 mg/mouse) versus the vehicle (i.e. 0.4% (v/v) tween 80 and 0.5% (w/v) carboxymethylcellulose in normal saline) and versus a comparator, i.e. CICATRENE cream (Johnson & Johnson), a drug product used in wound management and wound healing. The experimental plan is reported in Table 7.

TABLE 7
In vivo test of wound healing activity of cortexolone
17α-propionate - Experimental plan.

| | | | Route of | In vivo | Area of in vivo |
|---|---|---|---|---|---|
| | | Number of | administration | optical | photo-emission |
| Drug | Animals | Animals | and dosage | imaging | quantification |
| Vehicle | 6-8 weeks | 4 | Spread topically | Time 0, | Wound area |
| | male | | on the area of | 1, 2, 3, | |
| | REPTOP | | the wound | 6 days | |
| | mitoIRE | | (200 μl/mouse) | | |
| Cortexolone | | 4 | Spread topically | Time 0, | Wound area |
| 17α- | | | on the area of | 1, 2, 3, | |
| propionate | | | the wound | 6 days | |
| | | | (50 mg/mouse) | | |
| Cortexolone | | 4 | Spread topically | Time 0, | Wound area |
| 17α- | | | on the area of | 1, 2, 3, | |
| propionate | | | the wound | 6 days | |
| | | | (250 mg/mouse) | | |
| CICATRENE | | 4 | Spread topically | Time 0, | Wound area |
| | | | on the area of | 1, 2, 3, | |
| | | | the wound | 6 days | |
| | | | (200 mg/mouse) | | |

Animal Treatments.

The REPTOP mitolRE mice were shaved by VEET® depilatory cream a day before the treatment. A round full-thickness wound of 0.4 cm² area was made through the dorsal skin and the panniculus carnosus muscle of REPTOP mitolRE by using skin biopsy devices (HS biopsy punch).

In Vivo Test.

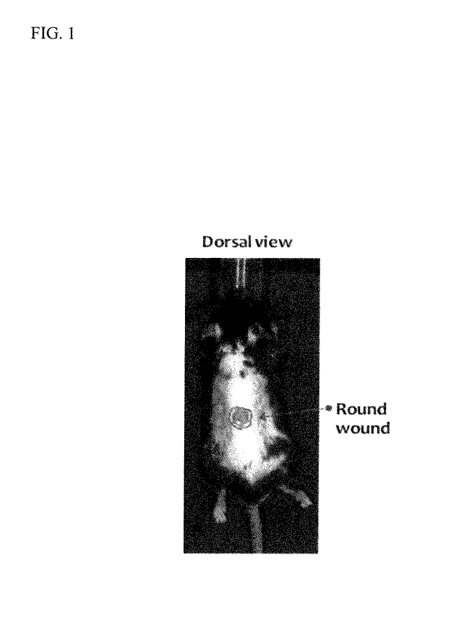

In order to establish the proliferative stimulating activity of cortexolone 17α-propionate, the vehicle (200 μl/mouse), cortexolone 17α-propionate (50 mg/mouse), cortexolone 17α-propionate (250 mg/mouse) and CICATRENE (200 mg/mouse) were spread topically on the wound of REPTOP mitolRE mice (4 mice per group of treatment), immediately after the generation of the round punch. Photon-emission was measured in wound area at the following times: time 0 and 1, 2, 3 and 6 days after the treatment. A representation of the area of wound selected for the measurements is depicted in FIG. 1.

In Vivo Imaging.

Animals were administered with 80 mg/kg of luciferin (Beetle Luciferin Potassium Salt; Promega, Madison, Wis., USA) by intraperitoneal injection 15 min before each imaging session. Mice were anaesthetized using isoflurane (ISOFLURAN-VET; Merial) and kept under anesthesia during the 5 minutes of the imaging session, which was carried out using a CCD-camera performing dorsal acquisitions (Xenogen IVIS Lumina System; Caliper, a PerkinElmer company). Photon emission in wound area was measured using the Living Image Software (Caliper, a PerkinElmer company) and expressed as photon/second/cm²/steradian (p/s/cm²/sr). The measurement of wound areas was expressed as cm².

Results.

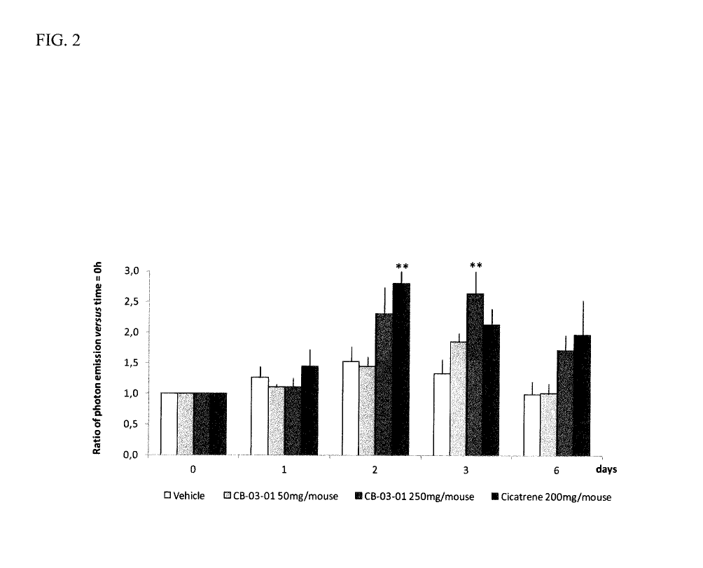

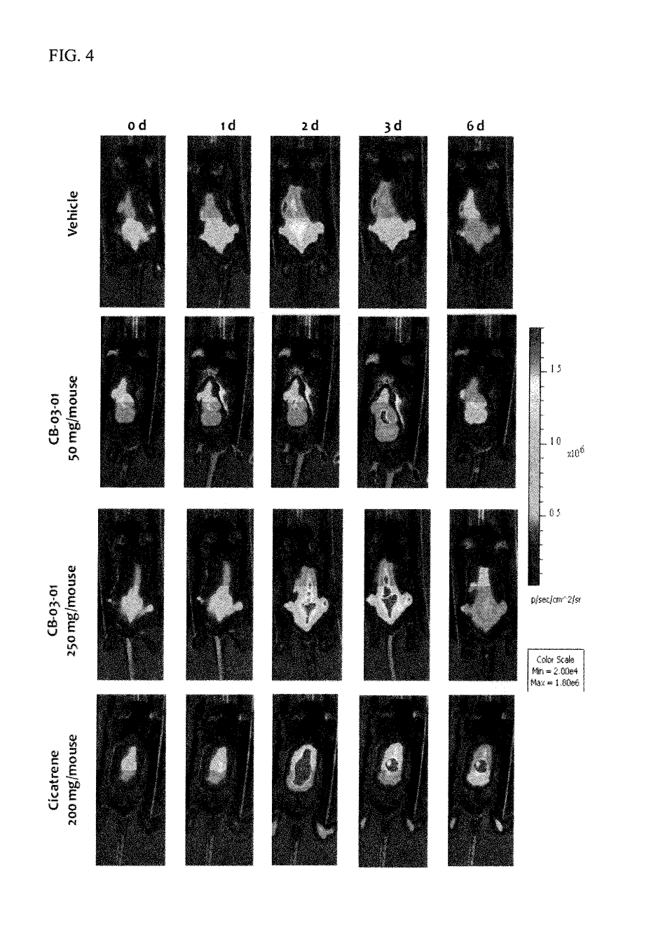

As shown in FIG. 2, in the days immediately after the treatment both cortexolone 17α-propionate (50 mg/mouse and 250 mg/mouse) and CICATRENE (200 mg/mouse) induce an increase of photon-emission from wound area. The CICATRENE treatment (200 mg/mouse) induces the maximum increase of proliferative cells after 2 days from administration; as a matter of facts, comparing time 2 days to time 0, a 131% increase of photon-emission from wounds can be observed (p<0.01 vs time 0). The cortexolone 17α-propionate treatment induces the maximum increase of proliferative cells after 3 days from administration: in facts, a 143% increase of photon-emission with respect to time 0 can be observed in cortexolone 17α-propionate 250 mg/mouse group (p<0.01 vs time 0). On the contrary, a very low effect is measured in animals treated with vehicle, since the maximum increase of photon-emission with respect to time 0 is 36% after 2 days. This observation confirms the hypothesis that cortexolone 17α-propionate induces a proliferative activity when spread onto the skin wound, and suggests that its maximal proliferative activity, non inferior to CICATRENE activity, shows a maximum after 3 days from the administration. FIG. 4 shows the in vivo wound healing bioluminescence imaging of REPTOP mitolRE mouse after indicated treatments.

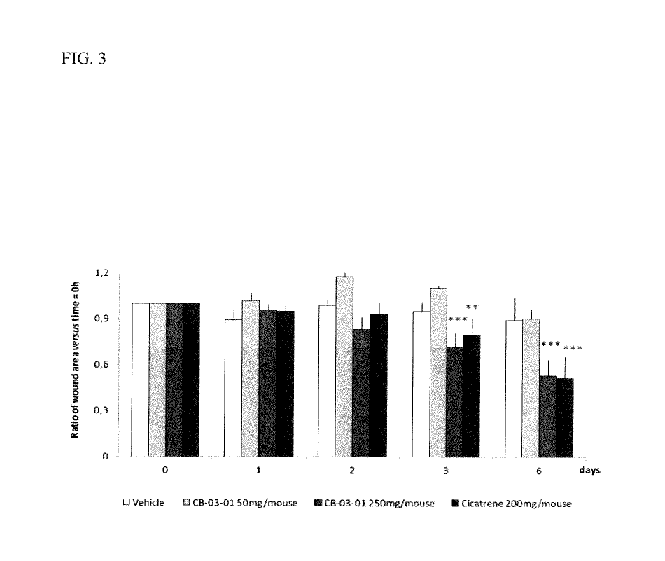

As shown in FIG. 3, a significant reduction of wound area was measured in the animals treated with 250 mg of cortexolone 17α-propionate (after 3 days p<0.001 vs time 0; after 6 days p<0.001 vs time 0) and in those treated with 200 mg of CICATRENE (after 3 days p<0.01 vs time 0; after 6 days p<0.001 vs time 0). After 3 days from administration, the mean reductions in wound area in the three treatment groups were: 19% for the animals treated with 200 mg of CICATRENE,_26% for the animals treated with 250 mg of cortexolone 17α-propionate and 6% for the animals treated with vehicle. These data suggest that cortexolone 17α-propionate (250 mg/mouse) accelerates wound healing respect to CICATRENE (200 mg/mouse) and respect to the vehicle.

### Example 6: Cortexolone 17α-Propionate Topical Powder

A topical powder composition comprising cortexolone 17α-propionate has been prepared by accurately mixing 100 g of the active ingredient finely sieved with 120 g of an absorbent powder, talc, and 30 g of a flow promoting agent, namely silicon dioxide. The resulting mixture underwent to sterilization by irradiation and then applied, in amount of approximately 50 mg, twice a daily to an ulcerated wound on the back of a mouse. The powder application immediate effect was the adsorption of the exudation liquid and the final effect was the healing of the wound within ten days.

### Example 7: Cortexolone 17α-Propionate Topical Solution

The same experiment described in the example 6 has been repeated with a liquid formulation obtained by accurately dissolving 50 g of cortexolone 17α-propionate in 300 g of propylene glycol and 150 g of polyethylene glycol 200 (PEG 200). The resulting solution has been filtered through a 0.22 μm membrane and bottled in individual vials of 30 g of capacity. The solution has been applied through a gauze to the back of the mouse every day for 5 days to promote the wound healing. The same solution has also been applied with a nozzle in the last 5 days by spraying 100 μL.

### Example 8: Cortexolone 17α-Propionate Topical Gel

The same experiment described in the example 6 has been repeated with a semisolid formulation obtained by accurately dissolving 100 g of cortexolone 17α-propionate in 500 g of propylene glycol and 98 g of isopropylmiristate, then adding 2 g of CARBOPOL 980 to make a viscous gel to be applied on the mouse back over the wound. The resulting gel has been packaged in aluminum tubes of 30 g of capacity. The gel has been applied to the back of the mouse every day for 10 days and resulted effective in promoting the wound healing.

### Example 9: Cortexolone 17α-Propionate Topical Ointment

The same experiment described in the example 6 has been repeated with a semisolid formulation obtained by accurately dispersing 30 g of cortexolone 17α-propionate, previously sterilized by irradiation, in 600 g of a sterile petrolatum-mineral oil ointment base, to make an ointment to be applied on the mouse back over the wound. The resulting cream has been packaged in blind end, aluminium tubes of 30 g of capacity provided with a fine tip. The resulting cream has been applied to the back of the mouse twice a day for 10 days and resulted effective in promoting the wound healing.

### Example 10: Cortexolone 17α-Propionate Topical Cream

| Component | (g) |
|---|---|
| Cortexolone 17α-propionate | 50.0 |
| Cetyl alcohol | 25.0 |
| Glyceryl monostearate | 150.0 |
| Liquid paraffin | 100.0 |
| Propylene glycol | 250.0 |
| Tocopherols | 0.5 |
| Sodium edetate | 1.0 |
| Polysorbate 80 | 10.0 |
| Purified water | 413.5 |

In a suitable vessel, 50.0 g of cortexolone 17α-propionate are dissolved in 250.0 g of propylene glycol under stirring (Solution A).

In another vessel, provided with a mechanical stirrer and a turboemulsifier, an emulsion containing 25.0 g of cetyl alcohol, 150.0 g of glyceryl monostearate, 100.0 g of liquid paraffin, 0.5 g of tocopherols, 1.0 g of sodium edetate, 10.0 g of polysorbate 80 and 413.5 g of purified water is prepared, operating at a temperature of about 70° C. The emulsion is cooled at about 30° C., then it is added with Solution A. The cream is maintained under stirring until homogeneity.

### Example 11: Cortexolone 17α-Propionate Topical Cream

| Component (g) | (g) |
|---|---|
| Cortexolone 17α-propionate | 30.0 |
| Mixture of glycerol monostearate and PEG-75 | 30.0 |
| Stearate (GELOT 64) | |
| Mixture of cetyl alcohol and ethoxylated fatty | 30.0 |
| alcohols (ceteth-20, steareth-20) (EMULCIRE 61WL2659) | |
| Cetyl alcohol | 30.0 |
| Caprylic/capric triglyceride | 120.0 |
| Diethylene glycol monoethyl ether (TRANSCUTOL) | 200.0 |
| Purified water | 560.0 |

In a suitable vessel, 30.0 g of cortexolone 17α-propionate are dissolved in 200.0 g of Diethylene glycol monoethyl ether (TRANSCUTOL) under stirring (Solution A).

In another vessel, provided with a mechanical stirrer and a turboemulsifier, an emulsion containing 30.0 g of GELOT 64, 30.0 g of EMULCIRE 61WL2659, 30.0 g of cetyl alcohol, 120.0 g of caprylic/capric triglyceride and 560.0 g of purified water is prepared, operating at a temperature of about 70° C. The emulsion is cooled at about 30° C., then it is added with Solution A. The cream is maintained under stirring until homogeneity.

### Example 12: Cortexolone 17α-Propionate Topical Gel

| Component | (g) |
|---|---|
| Cortexolone 17α-propionate | 10.0 |
| Caprylocapryl macro go I glycerides (LABRASOL) | 100.0 |
| Diethylene glycol monoethyl ether (TRANSCUTOL) | 220.0 |
| Hydroxyethyl cellulose | 20.0 |
| Purified water | 650.0 |

In a suitable vessel, 10.0 g of cortexolone 17α-propionate are dissolved in 220.0 g of diethylene glycol monoethyl ether (TRANSCUTOL) under stirring; then, 100.0 g of caprylocapryl macrogol glycerides (LABRASOL) are added and the mixture is stirred until homogeneity. To the obtained solution, 250.0 g of purified water are added under stirring; the stirrer is operated until a homogeneous emulsion is obtained (Emulsion A).

In another vessel, 20.0 g of hydroxyethyl cellulose are dissolved in 400.0 g of purified water under stirring, until a homogeneous gel is obtained. The gel is then added with Emulsion A and the gel is stirred until homogeneity.

### Example 13: Cortexolone 17α-Propionate Topical Gel

| Component | (g) |
|---|---|
| Cortexolone 17α-propionate | 30.0 |
| Caprylic/capric triglyceride | 570.0 |
| Ethyl cellulose | 60.0 |
| Oleoyl macrogol glycerides | 140.0 |
| Diethylene glycol monoethyl ether (TRANSCUTOL) | 200.0 |

In a suitable vessel, 30.0 g of cortexolone 17α-propionate are dissolved in 200.0 g of diethylene glycol monoethyl ether (TRANSCUTOL); the obtained solution is added to a mixture of 570.0 g of caprylic/capric triglyceride and 140.0 g of oleoyl macrogol glycerides. The mixture is stirred until homogeneity by means of a mechanical stirrer. 60.0 g of ethyl cellulose are added stepwise under stirring. The gel is maintained under stirring until homogeneity.

### Example 14: Cortexolone 17α-Propionate Topical Gel

| Component | (g) |
|---|---|
| Cortexolone 17α-propionate | 50.0 |
| Polyethylene glycol 200 (PEG 200) | 350.0 |
| Polyethylene glycol 400 (PEG 400) | 300.0 |
| Polyethylene glycol 1500 (PEG 1500) | 300.0 |

In a suitable vessel, 350.0 g of polyethylene glycol 200 (PEG 200), 300.0 g of polyethylene glycol 400 (PEG 400) and 300.0 g of polyethylene glycol 1500 (PEG 1500) are heated at 60° C. under stirring; the mixture is stirred at 60° C. until a clear solution is obtained. Then, the temperature is lowered to 30° C. and 50.0 g of cortexolone 17α-propionate are added. The gel is stirred until homogeneity.

### Example 15: Cortexolone 17α-Propionate Topical Spray

| Component | (g) |
|---|---|
| Cortexolone 17α-propionate | 20.0 |
| Caprylocapryl macrogol glycerides (LABRASOL) | 400.0 |
| Propylene glycol monolaurate | 60.0 |
| Oleoyl macrogol glycerides | 150.0 |
| Diethylene glycol monoethyl ether (TRANSCUTOL) | 200.0 |
| Glyceryl behenate | 120.0 |
| Purified water | 50.0 |

In a suitable vessel, 20.0 g of cortexolone 17α-propionate are dissolved in 200.0 g of diethylene glycol monoethyl ether (TRANSCUTOL) (Solution A).

In another vessel, provided with a mechanical stirrer and a turboemulsifier, 400.0 g caprylocapryl macrogol glycerides (LABRASOL), 60.0 g of propylene glycol monolaurate, 150.0 g of oleoyl macrogol glycerides and 120.0 g of glyceryl behenate are heated at 80° C. under stirring. Then, 50.0 g of purified water are added under stirring; the obtained emulsion is cooled at 30° C. and is added with Solution A under stirring. The mixture is stirred until homogeneity.

### Example 16: Cortexolone 17α-Propionate Topical Spray

| Component | (g) |
|---|---|
| Cortexolone 17α-propionate | 20.0 |
| Diethylene glycol monoethyl ether | 410.0 |
| (TRANSCUTOL) | |
| Propylene glycol | 560.0 |
| Copovidone | 10.0 |

In a suitable vessel, 20.0 g of cortexolone 17α-propionate are dissolved in 410.0 g of diethylene glycol monoethyl ether (TRANSCUTOL) and 560.0 g of propylene glycol. The solution is added with 10.0 g of copovidone and is kept under stirring until homogeneity.

The solution has been applied with a nozzle by spraying 100 μL, to the back of the mouse every day for 5 days to promote the wound healing.

### Example 17: Cortexolone 17α-Propionate Topical Powder

| Component | (g) |
|---|---|
| Cortexolone 17α-propionate | 50.0 |
| Sodium carboxymethyl cellulose | 390.0 |
| Talc | 250.0 |
| Kaolin | 300.0 |
| Silica, colloidal | 10.0 |

A topical powder composition comprising cortexolone 17α-propionate is prepared by accurately mixing 50.0 g of the active ingredient finely sieved with 390.0 g of sodium carboxymethyl cellulose, 250.0 g of talc, 300.0 g of kaolin and 10.0 g of colloidal silica. The resulting mixture is sterilized by irradiation.

### Example 18: Cortexolone 17α-Propionate Topical Powder

| Component | (g) |
|---|---|
| Cortexolone 17α-propionate | 150.0 |
| Chitosan | 390.0 |
| Hyaluronic acid | 1.0 |
| Aluminum magnesium silicate | 449.0 |
| Silica, colloidal | 10.0 |

A topical powder composition comprising cortexolone 17α-propionate is prepared by accurately mixing 150.0 g of the active ingredient finely sieved with 390.0 g of chitosan, 449.0 g of aluminium magnesium silicate, 1.0 g of hyaluronic acid and 10.0 g of colloidal silica. The resulting mixture is sterilized by irradiation.

The powder was applied, in amount of approximately 50 mg, twice a day to an ulcerated wound on the back of a mouse. The final effect was the healing of the wound within ten days.

### Example 19: Cortexolone 17α-Propionate Topical Gel

| Component | (g) |
|---|---|
| Cortexolone 17α-propionate | 30.0 |
| Propylene glycol | 679.0 |
| Ascorbyl Palmitate | 5.0 |
| Diethylene glycol monoethyl ether | 280.0 |
| Polysorbate | 1.0 |
| Hydroxypropylcellulose | 5.0 |

In a suitable vessel 30.0 g of cortexolone 17α-propionate are solubilized in a mixture of 280.0 g diethylene_glycol monoethyl ether and 679.0 g of propylene glycol. Then 5.0 g of ascorbyl palmitate and 1.0 g of polysorbate were added and dissolved under stirring. 5.0 g of hydroxypropylcellulose are homogenized to obtain a gel.

### Example 20: Cortexolone 17α-Propionate Topical Spray

| Component | (g) |
|---|---|
| Cortexolone 17α-propionate | 30.0 |
| Propylene glycol | 679.0 |
| Ascorbyl Palmitate | 5.0 |
| Diethylene glycol monoethyl ether | 280.0 |
| Polysorbate | 1.0 |
| Povidone | 15.0 |

In a suitable vessel 30.0 g of cortexolone 17α-propionate are solubilized in a mixture of 280.0 g diethylene glycol monoethyl ether and 679.0 g of propylene glycol. Then 5.0 g of ascorbyl palmitate and 1.0 g of polysorbate and 15.0 g of povidone were added and dissolved under stirring until homogeneous viscous solution is obtained.

### Example 21: Cortexolone 17α-Propionate Topical Spray

| Component | (g) |
|---|---|
| Cortexolone 17α-propionate | 30.0 |
| Propylene glycol | 675.0 |
| Ascorbyl Palmitate | 5.0 |
| Diethylene glycol monoethyl ether | 280.0 |
| Colloidal silicon dioxide | 10.0 |

In a suitable vessel, 30.0 g of cortexolone 17α-propionate are solubilized in a mixture of 280.0 g diethylene glycol monoethyl ether and 675.0 g of Propylene glycol. Then were added and dissolved 5.0 g of ascorbyl palmitate and 10.0 g of colloidal silicon dioxide are homogenized until homogeneous sprayable dispersion is obtained.

Several percentage of colloidal silicon dioxide are tested form 0.5% to 10% w/w

### Example 22: Cortexolone 17α-Propionate Topical Cream

| Component | (g) |
|---|---|
| Cortexolone 17α-propionate | 30.0 |
| Hard fat | 675.0 |
| Ascorbyl Palmitate | 5.0 |
| Diethylene glycol monoethyl ether | 280.0 |
| Colloidal silicon dioxide | 10.0 |

In a suitable jacketed vessel, melt 675.0 g of hard fat at 40° C. then dissolved 280.0 g diethylene glycol monoethyl ether then add and dissolve 30.0 g of cortexolone 17α-propionate then 5.0 g of ascorbyl palmitate and 10.0 g of colloidal silicon dioxide are homogenized until homogeneous sprayable dispersion is obtained.

### Example 23: Cortexolone 17α-Propionate Topical Powder

| Component | (g) |
|---|---|
| Cortexolone 17α-propionate | 30.0 |
| Poloxamer | 60.0 |
| Chitosan | 60.0 |
| Talc | 390.0 |
| Magnesium Oxide | 390.0 |
| Silica, colloidal | 50.0 |
| Simethicone | 20.0 |

A topical powder composition comprising cortexolone 17α-propionate is prepared by accurately mixing 30.0 g of the active ingredient finely sieved with 390.0 g of talc. 390.0 g of magnesium oxide 60.0 g of poloxamer. 60.0 g of chitosan and 20.0 of simethicone previously absorbed on 50.0 g of colloidal silicon dioxide. The resulting mixture is sterilized by irradiation.

### Example 24: Cortexolone 17α-Propionate Topical Spray

| Component | (g) |
|---|---|
| Cortexolone 17α-propionate | 30.0 |
| Propylene glycol | 534.0 |
| Ascorbyl Palmitate | 5.0 |
| Diethylene glycol monoethyl ether | 280.0 |
| Colloidal silicon dioxide | 50.0 |
| Xylitol | 50.0 |
| Polysorbate | 1.0 |
| Titanium dioxide | 50.0 |

In a suitable vessel, 280.0 g diethylene glycol monoethyl ether and 534.0 g of propylene glycol and 50.0 g of xylitol are mixed together under stirring at 40° C. Then 1.0 g of polysorbate, 5.0 g of ascorbyl palmitate, 50.0 g of colloidal silicon dioxide and 50.0 g titanium dioxide are added and homogenized until homogeneous dispersion is obtained; then 30.0 g of cortexolone 17α-propionate were solubilized under stirring until homogeneous sprayable dispersion is obtained.

### Example 25: Cortexolone 17α-Propionate Topical Powder

| Component | (g) |
|---|---|
| Cortexolone 17α-propionate | 30.0 |
| Poloxamer | 150.0 |
| Talc | 377.0 |
| β-Cyclodextrin | 73.0 |
| Silica colloidal dioxide | 50.0 |
| Simethicone | 20.0 |
| Magnesium Oxide | 300.0 |

A topical powder composition comprising cortexolone 17α-propionate is prepared by intimate mixing 30.0 g of the active ingredient finely sieved with β-cyclodextrin then mixed together with 377.0 g of talc, 300.0 g of magnesium oxide, 150.0 g of Poloxamer and 20.0 of simethicone previously absorbed on 50.0 g of silica colloidal dioxide. The resulting mixture is sterilized by irradiation.

### Example 26: Cortexolone 17α-Propionate Topical Powder

| Component | (g) |
|---|---|
| Cortexolone 17α-propionate | 30.0 |
| Poloxamer | 75.0 |
| Chitosan | 75.0 |
| Talc | 377.0 |
| β-Cyclodextrin | 73.0 |
| Silica colloidal | 50.0 |
| Maize Starch | 300.0 |

A topical powder composition comprising cortexolone 17α-propionate is prepared by intimate mixing 30.0 g of the active ingredient finely sieved with β-cyclodextrin, then mixed together with 377.0 g of talc, 300.0 g of maize starch, 75.0 g of poloxamer, 75.0 g of chitosan and 20.0 of simethicone previously absorbed on 50.0 g of colloidal silicon dioxide. The resulting mixture is sterilized by irradiation.

### Example 27: Cortexolone 17α-Propionate Topical Pressurized Spray

| Component | (g) |
|---|---|
| Cortexolone 17α-propionate | 30.0 |
| Propylene glycol | 384.0 |
| Ascorbyl Palmitate | 5.0 |
| Diethylene glycol monoethyl ether | 280.0 |
| Colloidal silicon dioxide | 50.0 |
| Sorbitol | 50.0 |
| Polysorbate | 1.0 |
| Carbon dioxide | 200.0 |

In a suitable vessel 280.0 g diethylene glycol monoethyl ether and 384.0 g of propylene glycol and 50.0 g of sorbitol are mixed together under stirring at 40° C. Then 5.0 g of ascorbyl palmitate, 1.0 g of polysorbate and 50.0 g of colloidal silicon dioxide are homogenized until homogeneous dispersion is obtained then 30.0 g of cortexolone 17α-propionate were solubilized under stirring until homogeneous dispersion is obtained. (Solution A).

The dispersion obtained. Solution A is packed in an aluminium bottle closed with an appropriate valve then the carbon dioxide as propellant is added in proportional quantity.

### Example 28: Cortexolone 17α-Propionate Topical Pressurized Spray

| Component | (g) |
|---|---|
| Cortexolone 17α-propionate | 30.0 |
| Propylene glycol | 384.0 |
| Ascorbyl Palmitate | 5.0 |
| Diethylene glycol monoethyl ether | 280.0 |
| Colloidal silicon dioxide | 50.0 |
| Xylitol | 50.0 |
| Polysorbate | 1.0 |
| Nitrogen | 200.0 |

In a suitable vessel, 280.0 g diethylene glycol monoethyl ether and 384.0 g of propylene glycol and 50.0 g of xylitol are mixed together under stirring at 40° C. Then 5.0 g of ascorbyl palmitate, 1.0 g of polysorbate and 50.0 g of colloidal silicon dioxide are homogenized until homogeneous dispersion is obtained then 30.0 g of cortexolone 17α-propionate were solubilized under stirring until homogeneous dispersion is obtained. (Solution A).

The dispersion obtained. Solution A is packed in an aluminium bottle closed with an appropriate valve then the Nitrogen as propellant is added in proportional quantity.

### Example 29: Cortexolone 17α-Propionate Pressurized Topical Foam

| Component | (g) |
|---|---|
| Cortexolone 17α-propionate | 30.0 |
| Propylene glycol | 200.0 |
| Diethylene glycol monoethyl ether | 280.0 |
| Polysorbate | 10.0 |
| Ascorbyl Palmitate | 5.0 |
| Butane Propane | 475.0 |

In a suitable vessel, 280.0 g diethylene glycol monoethyl ether and 200.0 g of propylene glycol are mixed together under stirring. Then 5.0 g of ascorbyl palmitate, 10.0 g of polysorbate are added and dissolved under stirring until homogeneous dispersion is obtained then 30.0 g of cortexolone 17α-propionate is solubilized under stirring until homogeneous dispersion is obtained. (Solution A). The dispersion obtained Solution A is packed in an aluminium bottle closed with an appropriate valve then the butane propane as propellant is added in proportional quantity.

### Example 30: Cortexolone 17α-Propionate Pressurized Topical Foam

| Component | (g) |
|---|---|
| Cortexolone 17α-propionate | 30.0 |
| Propylene glycol | 200.0 |
| Diethylene glycol monoethyl ether | 280.0 |
| Polysorbate | 10.0 |
| Ascorbyl Palmitate | 5.0 |
| Simethicone | 1.0 |
| Mannitol | 50.0 |
| Butane Propane | 424.0 |

In a suitable vessel 280.0 g diethylene glycol monoethyl ether and 200.0 g of propylene glycol and 50.0 g of mannitol are mixed together under stirring at 40° C. Then 5.0 g of ascorbyl palmitate. 10.0 g of polysorbate and 1.0 g of simethicone are mixed under stirring until an homogeneous dispersion is obtained then 30.0 g of cortexolone 17α-propionate were solubilized under stirring until homogeneous dispersion is obtained. (Solution A).

The dispersion obtained. Solution A. is packed in an aluminium bottle closed with an appropriate valve then the butane propane as propellant is added.

What is claimed is:

1. A method of treating at least one skin wound and/or at least one atrophic skin disorder, comprising administering to a subject in need thereof, an effective amount of cortexolone 17α-propionate.

2. The method according to claim 1, wherein said at least one skin wound is selected from open skin wounds, burn skin wounds, and/or chronic skin wounds.

3. The method according to claim 2, wherein said open skin wounds are selected from incisions, incised wounds, surgical wounds, lacerations, abrasions, puncture wounds, bite wounds, scratch wounds, penetration wounds, gunshot wounds, avulsions, and/or blisters.

4. The method according to claim 2, wherein said burn skin wounds are selected from burn wounds caused by heat, burn wounds caused by friction, burn wounds caused by electricity, burn wounds caused by chemicals, and/or burn wounds caused by radiation.

5. The method according to claim 2, wherein said chronic skin wounds are selected from cutaneous ulcers, trophic ulcers of the skin, radiation injuries, and/or chronic ulcers in elderly humans.

6. The method according to claim 5, wherein said trophic ulcers of the skin are selected from decubitus ulcers, lower-extremity ulcers, diabetic ulcers, neuropathic ulcers, venous stasis ulcers, arterial ulcers, and/or diabetic foot ulcers.

7. The method according to claim 1, wherein said at least one atrophic skin disorder is selected from skin ageing, photo-ageing, wrinkles, lines, dermatomyositis, atrophic striae, radiation dermatitis, scars, acrodermatitis, and/or anetoderma.

8. The method according to claim 1, wherein said cortexolone 17α-propionate is administered to a mammal.

9. The method according to claim 8, wherein said mammal is a human.

10. The method according to claim 8, wherein said mammal is an animal.

11. The method according to claim 10, wherein said animal is selected from canids, felines, bovines, bovids, ovines, equines, and/or swines.

12. The method according to claim 1, wherein said cortexolone 17α-propionate is administered topically.

13. The method according to claim 12, wherein said cortexolone 17α-propionate is administered in a solid, semi-solid, pasty, or liquid form.

14. The method according to claim 13, wherein said method is for treating burn skin wounds or trophic skin ulcers.

15. A method of treating at least one skin wound and/or at least one atrophic skin disorder, comprising administering to a subject in need thereof a pharmaceutical or cosmetic composition comprising an effective amount of cortexolone 17α-propionate and at least one physiologically acceptable excipient.

16. The method according to claim 15, wherein said method is for treating burn skin wounds or trophic skin ulcers.

17. The method according to claim 15, wherein said composition is in a solid, semi-solid, pasty, or liquid form.

18. The method according to claim 17, wherein said composition is formulated in a plaster, a gauze, or a medicated gauze.

19. The method according to claim 17, wherein said composition is selected from the group consisting of a powder, a freeze-dried powder, a solution, an emulsion, a gel, a paste, a cream, an ointment, a lotion, a suspension, a spray, and a pressurized spray.

20. The method according claim 19, wherein said composition is in a solid form and comprises cortexolone 17α-propionate in an amount ranging from about 0.1% w/w to about 80% w/w with respect to the total weight of the composition.

21. The method according to claim 20, wherein said composition comprises cortexolone 17α-propionate in an amount ranging from about 1% w/w to about 75% w/w with respect to the total weight of the composition.

22. The method according to claim 21, wherein said composition comprises cortexolone 17α-propionate in an amount ranging from about 5% w/w to about 70% w/w with respect to the total weight of the composition.

23. The method according to claim 22, wherein said composition comprises cortexolone 17α-propionate in an amount ranging from about 10% w/w to about 60% w/w with respect to the total weight of the composition.

24. The method according to claim 19, wherein said composition is in a semi-solid, pasty or liquid form and comprises cortexolone 17α-propionate in an amount ranging from about 0.1% w/w to about 50% w/w with respect to the total weight of the composition.

25. The method according to claim 24, wherein said composition comprises cortexolone 17α-propionate in an amount ranging from about 0.2% w/w to about 30% w/w with respect to the total weight of the composition.

26. The method according to claim 25, wherein said composition comprises cortexolone 17α-propionate in an amount ranging from about 0.5% w/w to about 20% w/w with respect to the total weight of the composition.

27. The method according to claim 26, wherein said composition comprises cortexolone 17α-propionate in an amount ranging from about 1% w/w to about 15% w/w with respect to the total weight of the composition.

28. The method according to claim 15, wherein said composition is administered to a mammal.

29. The method according to claim 28, wherein said mammal is a human.

30. The method according to claim 28, wherein said mammal is an animal.

31. The method according to claim 30, wherein said animal is selected from canids, felines, bovines, bovids, ovines, equines, and/or swines.
